(19) United States
(12) Patent Application Publication
(10) Pub. No.: US 2011/0195997 A1
(43) Pub. Date: Aug. 11, 2011

(54) Dicarboxamide Derivatives

(76) Inventors: Katrin Groebke Zbinden, Liestal (CH); Wolfgang Haap, Loerrach (DE); Hans Hilpert, Muenchenstein (CH); Narendra Panday, Muenchen (DE); Fabienne Ricklin, Hombourg (FR)

(21) Appl. No.: 13/088,459

(22) Filed: Apr. 18, 2011

Related U.S. Application Data
(63) Continuation of application No. 11/786,272, filed on Apr. 11, 2007, now abandoned.

(30) Foreign Application Priority Data
Apr. 21, 2006 (EP) 06112897.1

(51) Int. Cl. A61K 31/444; C07D 401/12; A61P 7/02

(52) U.S. Cl. 514/335; 546/261

(57) ABSTRACT

The invention is concerned with dicarboxamide derivatives of formula (I)

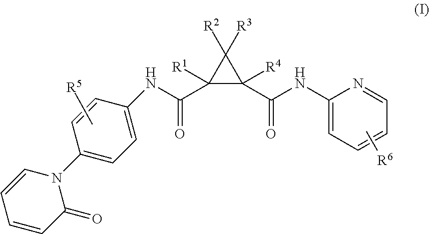

wherein R¹, R², R³, R⁴, R⁵ and R⁶ are as defined in the specification, as well as physiologically acceptable salts thereof. These compounds inhibit the coagulation factor Xa and can be used in pharmaceutical compositions.

### PRIORITY TO RELATED APPLICATIONS

This application is a continuation of U.S. application Ser. No. 11/786,272, filed Apr. 11, 2007, which claims the benefit of European Application No. 06112897.1, filed Apr. 21, 2006, which are hereby incorporated by reference in their entirety.

### BACKGROUND OF THE INVENTION

Factor Xa is a serine endopeptidase composed of two disulfide-linked subunits that converts prothrombin to thrombin in the blood coagulation cascade. It acts by cleaving prothrombin in two places (an arg-thr and then an arg-ile bond), which yields the active thrombin. Several inhibitors of factor Xa, which are not structurally related to the compounds of the present invention, had previously been suggested for the inhibition of the formation of thrombi and for the treatment of related diseases (WO 03/045912). However, there is still a need for novel factor Xa inhibitors which exhibit improved pharmacological properties, e.g. an improved selectivity towards coagulation factor Xa. The present invention relates to the novel compounds which are factor Xa inhibitors. The compounds of the present invention unexpectedly inhibit coagulation factor Xa and also exhibit improved pharmacological properties compared to other compounds already known in the art.

### SUMMARY OF THE INVENTION

The invention is directed to the dicarboxamide derivatives of formula (I) and all pharmaceutically acceptable salts thereof wherein formula (I) is:

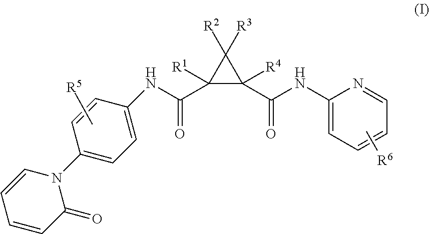

wherein R1-R6 are as described hereinafter in the Detailed Description of the Invention.

The invention is also directed to a process for the manufacture of such compounds, pharmaceutical compositions which contain such compounds as well as the use of such compounds for the treatment and/or prevention of thrombotic disorders.

The compounds of the present invention are active compounds which inhibit the coagulation factor Xa. These compounds consequently influence blood coagulation. They therefore inhibit the formation of thrombin and can be used for the treatment and/or prevention of thrombotic disorders, such as amongst others, arterial and venous thrombosis, deep vein thrombosis, peripheral arterial occlusive disease (PAOD), unstable angina pectoris, myocardial infarction, coronary artery disease, pulmonary embolism, stroke (cerebral thrombosis) due to atrial fibrillation, inflammation and arteriosclerosis. They have potential benefit for the treatment of acute vessel closure associated with thrombolytic therapy and restenosis, e.g. after transluminal coronary angioplasty (PTCA) or bypass grafting of the coronary or peripheral arteries and in the maintenance of vascular access patency in long term hemodialysis patients. The factor Xa inhibitors of this invention may form part of a combination therapy with an anticoagulant with a different mode of action or with a platelet aggregation inhibitor or with a thrombolytic agent. Furthermore, these compounds have an effect on tumor cells and prevent metastases. They can therefore also be used as antitumor agents.

### DETAILED DESCRIPTION OF THE INVENTION

The invention is directed to the compounds of formula (I) and all pharmaceutically acceptable salts thereof wherein formula (I) is:

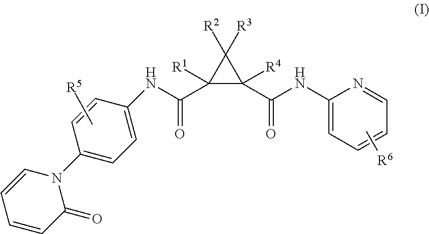

wherein:
(a) R² and R³ are independently from each other selected from the group consisting of:
   (1) hydrogen,
   (2) C₁₋₆ alkyl,
   (3) carboxyl,
   (4) C₁₋₆ alkoxycarbonyl,
   (5) carbamoyl,
   (6) mono-substituted amino-carbonyl or di-substituted amino-carbonyl,
   (7) optionally substituted arylcarbonyl,
   (8) optionally substituted heterocyclylcarbonyl,
   (9) optionally substituted heteroarylcarbonyl,
   (10) optionally substituted aryl,
   (11) optionally substituted heteroaryl,
   (12) optionally substituted heterocyclyl,
   (13) hydroxy C₁₋₆ alkyl,
   (14) halo C₁₋₆ alkyl,
   (15) cyano C₁₋₆ alkyl,
   (16) C₁₋₆ alkoxy C₁₋₆ alkyl,
   (17) amino C₁₋₆ alkyl,
   (18) mono-substituted or di-substituted amino-C₁₋₆ alkyl,
   (19) optionally substituted aryl C₁₋₆ alkyl,
   (20) optionally substituted heterocyclyl C₁₋₆ alkyl,
   (21) optionally substituted heteroaryl C₁₋₆ alkyl,
   (22) optionally substituted aryl C₁₋₆ alkoxy C₁₋₆ alkyl,
   (23) optionally substituted heteroaryl C₁₋₆ alkoxy C₁₋₆ alkyl, and
   (24) optionally substituted heterocyclyl C₁₋₆ alkoxy C₁₋₆ alkyl;
(b) R¹ and R⁴ are independently from each other selected from the group consisting of:
   (1) hydrogen,
   (2) C₁₋₆ alkyl,
   (3) cyano,
   (4) C₁₋₆ alkoxycarbonyl,
   (5) C₂₋₆ alkenyloxycarbonyl,
   (6) C₂₋₆ alkynyloxycarbonyl,
   (7) hydroxyl C₁₋₆ alkyl,
   (8) carboxyl,
   (9) mono- or di-C₁₋₆ alkyl substituted amino-carbonyl,
   (10) aminocarbonyl,
   (11) optionally substituted heterocyclylcarbonyl,
   (12) optionally substituted heteroarylcarbonyl, and
   (13) optionally substituted arylcarbonyl; and
(c) R⁵ and R⁶ are independently from each other selected from the group consisting of: chlorine, fluorine, and bromine.

In a preferred embodiment, R² and R³ are independently from each other selected from the group consisting of:

(1) hydrogen,

(2) C₁₋₆ alkyl,

(3) carboxyl,

(4) C₁₋₆ alkoxycarbonyl,

(5) carbamoyl,

(6) mono-substituted or di-substituted amino-carbonyl,

(7) optionally substituted heterocyclylcarbonyl,

(8) optionally substituted heteroarylcarbonyl,

(9) aryl,

(10) optionally substituted heteroaryl,

(11) optionally substituted heterocyclyl,

(12) hydroxyl C₁₋₆ alkyl,

(13) C₁₋₆ alkoxy C₁₋₆ alkyl,

(14) amino C₁₋₆ alkyl,

(15) mono-substituted or di-substituted amino-C₁₋₆ alkyl,

(16) optionally substituted heterocyclyl C₁₋₆ alkyl, and

(17) optionally substituted heteroaryl C₁₋₆ alkyl.

In a preferred embodiment, R¹ and R⁴ are independently from each other selected from the group consisting of: hydrogen and C₁₋₆ alkyl.

The compounds of the present invention can form pharmaceutically acceptable acid addition salts. Examples of such pharmaceutically acceptable salts are salts of the compounds with physiologically compatible mineral acids, such as hydrochloric acid, sulphuric acid, sulphurous acid or phosphoric acid; or with organic acids, such as methanesulphonic acid, p-toluenesulphonic acid, acetic acid, lactic acid, trifluoroacetic acid, citric acid, fumaric acid, maleic acid, tartaric acid, succinic acid or salicylic acid. The term “pharmaceutically acceptable salts” includes such salts. Acid addition salts as described above are preferred.

Unless otherwise indicated, the following definitions are set forth to illustrate and define the meaning and scope of the various terms used to describe the invention herein.

The term “C₁₋₆ alkyl” means a branched or straight-chain monovalent alkyl radical, having one to six carbon atoms. This term is further exemplified by such radicals as methyl, ethyl, n-propyl, isopropyl, n-butyl, s-butyl, t-butyl. Methyl is more preferred.

The term“halo C₁₋₆ alkyl” means a C₁₋₆ alkyl substituted by one or more halogen atoms independently selected from the group consisting of chlorine, fluorine and bromine.

The term “cyano C₁₋₆ alkyl” means a C₁₋₆ alkyl substituted by one or more cyano groups. In a preferred embodiment the cyano C₁₋₆ alkyl is substituted by one cyano group.

The term “hydroxy C₁₋₆ alkyl” means a C₁₋₆ alkyl substituted by one or more hydroxy groups. In a preferred embodiment the hydroxy C₁₋₆ alkyl is substituted by one or two hydroxy groups.

The term “C₃₋₇ cycloalkyl,” alone or in combination with other groups, means a saturated monovalent cyclic hydrocarbon radical of three to seven ring carbons (such as, for example, cyclopropyl, cyclobutyl, or cyclohexyl).

The term “C₁₋₆ alkoxy,” alone or in combination with other groups, means the group R′-O—, wherein R′ is a C₁₋₆ alkyl.

The term “C₂₋₆ alkenyl,” alone or in combination with other groups, means a straight-chain or branched hydrocarbon residue comprising an olefinic bond, having two to six carbon atoms (such as, for example, ethenyl or 2-propenyl).

The term “C₂₋₆ alkynyl,” alone or in combination with other groups, means a straight-chain or branched hydrocarbon residue comprising a triple bond and 2 to 6 carbon atoms, such as e.g. 2-propinyl.

The term “aryl,” alone or in combination with other groups, means a phenyl group or a naphthyl group. In a preferred embodiment the aryl is a phenyl group.

The term “optionally substituted aryl” means an aryl group described above, which is optionally substituted by one to five substituents independently selected from the group consisting of halogen, hydroxy, C₁₋₆ alkyl, halo C₁₋₆ alkyl, C₁₋₆ alkoxy, C₁₋₆ alkyl sulfonyl, C₁₋₆ alkyl sulfinyl, C₁₋₆ alkylthio, amino, amino C₁₋₆ alkyl, mono- or di-substituted amino-C₁₋₆ alkyl, nitro, cyano, acyl, carbamoyl, mono- or di-substituted amino, aminocarbonyl, mono- or di-substituted amino-carbonyl, aminocarbonyl C₁₋₆ alkoxy, mono- or di-substituted amino-carbonyl-C₁₋₆ alkoxy, hydroxy-C₁₋₆ alkyl, carboxyl, C₁₋₆ alkoxy carbonyl, aryl C₁₋₆ alkoxy, heteroaryl C₁₋₆ alkoxy, heterocyclyl C₁₋₆ alkoxy, C₁₋₆ alkoxycarbonyl C₁₋₆ alkoxy, carbamoyl C₁₋₆ alkoxy, and carboxyl C₁₋₆ alkoxy. In a preferred embodiment, the optionally substituted aryl is optionally substituted by one to three substituents. In another preferred embodiment, the optionally substituted aryl is optionally substituted by one to five substituents selected from the group consisting of halogen, hydroxy, C₁₋₆ alkyl, halo C₁₋₆ alkyl, C₁₋₆ alkoxy, C₁₋₆ alkyl sulfonyl, C₁₋₆ alkyl sulfinyl, C₁₋₆ alkylthio, amino, mono-C₁₋₆ alkyl substituted amino, di-C₁₋₆ alkyl substituted amino, amino C₁₋₆ alkyl, mono-C₁₋₆ alkyl substituted amino-C₁₋₆ alkyl, di-C₁₋₆ alkyl substituted amino-C₁₋₆ alkyl, nitro, and cyano.

The term “heterocyclyl,” alone or in combination with other groups, means a non-aromatic monocyclic radical of three to eight ring atoms in which one or two ring atoms are heteroatoms independently selected from the group consisting of N, O, and S(O)ₙ (where n is an integer from 0 to 2); with the remaining ring atoms being carbon atoms, wherein one or two ring carbon atoms of the heterocyclyl may be in the form of a carbonyl group.

The term “optionally substituted heterocyclyl” means a heterocyclyl group as described above, which is optionally substituted independently by one, two, or three substituents, selected from the group consisting of halogen, hydroxy, C₁₋₆ alkyl, halo C₁₋₆ alkyl, C₁₋₆ alkoxy, C₁₋₆ alkyl sulfonyl, C₁₋₆ alkyl sulfinyl, C₁₋₆ alkylthio, amino, amino C₁₋₆ alkyl, mono- or di-substituted amino-C₁₋₆ alkyl, nitro, cyano, acyl, carbamoyl, mono- or di-substituted amino, aminocarbonyl, mono- or di-substituted amino-carbonyl, aminocarbonyl C₁₋₆ alkoxy, mono- or di-substituted amino-carbonyl-C₁₋₆ alkoxy, hydroxy-C₁₋₆ alkyl, carboxyl, C₁₋₆ alkoxy carbonyl, aryl C₁₋₆ alkoxy, heteroaryl C₁₋₆ alkoxy, heterocyclyl C₁₋₆ alkoxy, C₁₋₆ alkoxycarbonyl C₁₋₆ alkoxy, carbamoyl C₁₋₆ alkoxy, and carboxyl C₁₋₆ alkoxy. In a preferred embodiment, the optionally substituted heterocyclyl is optionally substituted by one or two substituents. In another preferred embodiment the optionally substituted heterocyclyl is optionally substituted by one, two, or three substituents, independently selected from the group consisting of halogen, hydroxy, C₁₋₆ alkyl, halo C₁₋₆ alkyl, C₁₋₆ alkoxy, acyl, C₁₋₆ alkyl sulfonyl, C₁₋₆ alkyl sulfinyl, C₁₋₆ alkylthio, amino, mono-C₁₋₆ alkyl substituted amino, di-C₁₋₆ alkyl substituted amino, amino C₁₋₆ alkyl, mono-C₁₋₆ alkyl substituted amino-C₁₋₆ alkyl, di-C₁₋₆ alkyl substituted amino-C₁₋₆ alkyl, nitro, carbamoyl, mono- or di-substituted amino-carbonyl, hydroxy-C₁₋₆ alkyl, carboxyl, C₁₋₆ alkoxy carbonyl and cyano. In a more preferred embodiment, the optionally substituted heterocyclyl is optionally substituted by one, two, or three substituents, independently selected from the group consisting of halogen, hydroxy, C₁₋₆ alkyl, halo C₁₋₆ alkyl, C₁₋₆ alkoxy, C₁₋₆ alkyl sulfonyl, C₁₋₆ alkyl sulfinyl, C₁₋₆ alkylthio, amino, mono-C₁₋₆ alkyl substituted amino, di-C₁₋₆ alkyl substituted amino, amino C₁₋₆ alkyl, mono-C₁₋₆ alkyl substituted amino-C₁₋₆ alkyl, di-C₁₋₆ alkyl substituted amino-C₁₋₆ alkyl, nitro, and cyano.

The term “heteroaryl,” alone or in combination with other groups, means a monocyclic or bicyclic radical of 5 to 12 ring atoms having at least one aromatic ring containing one, two, or three ring heteroatoms independently selected from the group consisting of N, O, and S, with the remaining ring atoms being carbon atoms, with the proviso that the attachment point of the heteroaryl radical will be on the aromatic portion of the heteroaryl (i.e., on the aromatic ring of a bicyclic heteroaryl which has only one aromatic ring). One or two ring carbon atoms of the heteroaryl may be in the form of a carbonyl group.

The term “optionally substituted heteroaryl” means a heteroaryl group as described above, which is optionally substituted independently with one, two, or three substituents selected from the group consisting of halogen, hydroxy, C₁₋₆ alkyl, halo C₁₋₆ alkyl, C₁₋₆ alkoxy, C₁₋₆ alkyl sulfonyl, C₁₋₆ alkyl sulfinyl, C₁₋₆ alkylthio, amino, amino C₁₋₆ alkyl, mono- or di-substituted amino-C₁₋₆ alkyl, nitro, cyano, acyl, carbamoyl, mono- or di-substituted amino, aminocarbonyl, mono- or di-substituted amino-carbonyl, aminocarbonyl C₁₋₆ alkoxy, mono- or di-substituted amino-carbonyl-C₁₋₆ alkoxy, hydroxy-C₁₋₆ alkyl, carboxyl, C₁₋₆ alkoxy carbonyl, aryl C₁₋₆ alkoxy, heteroaryl C₁₋₆ alkoxy, heterocyclyl C₁₋₆ alkoxy, C₁₋₆ alkoxycarbonyl C₁₋₆ alkoxy, carbamoyl C₁₋₆ alkoxy, and carboxyl C₁₋₆ alkoxy. In a preferred embodiment, the optionally substituted heteroaryl is optionally substituted by one or two substituents. In another preferred embodiment the optionally substituted heteroaryl is optionally substituted by one, two, or three substituents selected from the group consisting of halogen, hydroxy, C₁₋₆ alkyl, halo C₁₋₆ alkyl, C₁₋₆ alkoxy, C₁₋₆ alkyl sulfonyl, C₁₋₆ alkyl sulfinyl, C₁₋₆ alkylthio, amino, mono-C₁₋₆ alkyl substituted amino, di-C₁₋₆ alkyl substituted amino, amino C₁₋₆ alkyl, mono-C₁₋₆ alkyl substituted amino-C₁₋₆ alkyl, di-C₁₋₆ alkyl substituted amino-C₁₋₆ alkyl, nitro, carbamoyl, mono- or di-substituted amino-carbonyl, hydroxy-C₁₋₆ alkyl, carboxyl, C₁₋₆ alkoxy carbonyl, and cyano.

The term “optionally substituted phenyl” means a phenyl group optionally substituted by one to five substituents, independently selected from the group consisting of halogen, hydroxy, C₁₋₆ alkyl, halo C₁₋₆ alkyl, C₁₋₆ alkoxy, C₁₋₆ alkyl sulfonyl, C₁₋₆ alkyl sulfinyl, C₁₋₆ alkylthio, amino, amino C₁₋₆ alkyl, mono- or di-substituted amino-C₁₋₆ alkyl, nitro, cyano, acyl, carbamoyl, mono- or di-substituted amino, aminocarbonyl, mono- or di-substituted amino-carbonyl, aminocarbonyl C₁₋₆ alkoxy, mono- or di-substituted amino-carbonyl-C₁₋₆ alkoxy, hydroxy-C₁₋₆ alkyl, carboxyl, C₁₋₆ alkoxy carbonyl, aryl C₁₋₆ alkoxy, heteroaryl C₁₋₆ alkoxy, heterocyclyl C₁₋₆ alkoxy, C₁₋₆ alkoxycarbonyl C₁₋₆ alkoxy, carbamoyl C₁₋₆ alkoxy, and carboxyl C₁₋₆ alkoxy. In a preferred embodiment, the optionally substituted phenyl is optionally substituted by one to three substituents. In another preferred embodiment the optionally substituted phenyl is optionally substituted by one to five substituents independently selected from the group consisting of halogen, hydroxy, C₁₋₆ alkyl, halo C₁₋₆ alkyl, C₁₋₆ alkoxy, C₁₋₆ alkyl sulfonyl, C₁₋₆ alkyl sulfinyl, C₁₋₆ alkylthio, amino, mono-C₁₋₆ alkyl substituted amino, di-C₁₋₆ alkyl substituted amino, amino C₁₋₆ alkyl, mono-C₁₋₆ alkyl substituted amino-C₁₋₆ alkyl, di-C₁₋₆ alkyl substituted amino-C₁₋₆ alkyl, nitro, and cyano.

The term “mono-substituted amino” and “di-substituted amino,” alone or in combination with other groups, mean respectively —NHR and —NRR′, in which R and R′ are independently selected from the group consisting of hydroxy, C₁₋₆ alkyl, hydroxy C₁₋₆ alkyl, C₁₋₆ alkoxy C₁₋₆ alkyl, carbamoyl C₁₋₆ alkyl, halo C₁₋₆ alkyl, C₃₋₇ cycloalkyl, C₃₋₇ cycloalkyl C₁₋₆ alkyl, C₁₋₆ alkoxy, C₁₋₆ alkyl sulfonyl, C₁₋₆ alkyl sulfinyl, C₁₋₆ alkylthio, mono- or di-C₁₋₆ alkyl substituted amino-sulfonyl, mono- or di-C₁₋₆ alkyl substituted amino-sulfinyl, mono- or di-C₁₋₆ alkyl substituted amino-thio, mono- or di-C₁₋₆ alkyl substituted amino-C₁₋₆ alkyl, mono- or di-C₁₋₆ alkyl substituted aminocarbonyl-C₁₋₆ alkyl, acyl, halo C₁₋₆ alkylcarbonyl, and C₁₋₆ alkoxycarbonyl. In a preferred embodiment, R and R′ are independently selected from the group consisting of hydroxy, C₁₋₆ alkyl, hydroxy C₁₋₆ alkyl, C₁₋₆ alkoxy C₁₋₆ alkyl, carbamoyl C₁₋₆ alkyl, halo C₁₋₆ alkyl, C₃₋₇ cycloalkyl, C₃₋₇ cycloalkyl C₁₋₆ alkyl, C₁₋₆ alkoxy, C₁₋₆ alkyl sulfonyl, C₁₋₆ alkyl sulfinyl, C₁₋₆ alkylthio, mono- or di-C₁₋₆ alkyl substituted amino-sulfonyl, mono- or di-C₁₋₆ alkyl substituted amino-sulfinyl, mono- or di-C₁₋₆ alkyl substituted amino-thio, mono- or di-C₁₋₆ alkyl substituted amino-C₁₋₆ alkyl, mono- or di-C₁₋₆ alkyl substituted aminocarbonyl-C₁₋₆ alkyl, acyl, and C₁₋₆ alkoxycarbonyl. In another preferred embodiment, R and R′ are independently selected from the group consisting of hydroxy, C₁₋₆ alkyl, hydroxy C₁₋₆ alkyl, halo C₁₋₆ alkyl, C₃₋₇ cycloalkyl, C₃₋₇ cycloalkyl C₁₋₆ alkyl, C₁₋₆ alkoxy, C₁₋₆ alkyl sulfonyl, C₁₋₆ alkyl sulfinyl, C₁₋₆ alkylthio, mono- or di-C₁₋₆ alkyl substituted amino-sulfonyl, mono- or di-C₁₋₆ alkyl substituted amino-sulfinyl, mono- or di-C₁₋₆ alkyl substituted amino-thio, acyl, and C₁₋₆ alkoxycarbonyl.

The term “acyl,” alone or in combination with other groups, means —C(═O)R, in which R is H or C₁₋₆ alkyl.

Preferred radicals for the chemical groups whose definitions are given above are those specifically exemplified in Examples.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the specification and claims includes both one or more than one such excipient.

As used herein, a “pharmaceutically acceptable carrier” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention are contemplated. Supplementary active compounds can also be incorporated into the compositions.

As used herein, the term “a therapeutically effective amount” of a compound means an amount of compound that is effective to prevent, alleviate or ameliorate symptoms of disease or prolong the survival of the subject being treated. Determination of a therapeutically effective amount is within the skill in the art.

The therapeutically effective amount or dosage of a compound according to this invention can vary within wide limits and may be determined in a manner known in the art. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 1 mg to about 10,000 mg, preferably from about 10 mg to about 1,000 mg, should be appropriate, although the upper limit may be exceeded when indicated. The daily dosage can be administered as a single dose or in divided doses, or for parenteral administration, it may be given as continuous infusion.

Compounds that have the same molecular Formula but differ in the nature or sequence of bonding of their atoms or the arrangement of their atoms in space are termed “isomers.” Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, if a carbon atom is bonded to four different groups, a pair of enantiomers is possible. An enantiomer can be characterized by the absolute configuration of its asymmetric center and is described by the R- and S-sequencing rules of Cahn, Ingold and Prelog, or by the manner in which the molecule rotates the plane of polarized light and designated as dextrorotatory or levorotatory (i.e., as (+) or (−)-isomers respectively). A chiral compound can exist as either an individual enantiomer or as a mixture thereof. A mixture containing equal proportions of the enantiomers is called a “racemic mixture.”

As described above, the compounds of the present invention are active compounds and inhibit the coagulation factor Xa. These compounds consequently influence both platelet activation which is induced by this factors and plasmatic blood coagulation. They therefore inhibit the formation of thrombin and can be used for the treatment and/or prevention of thrombotic disorders, such as, amongst others, arterial and venous thrombosis, deep vein thrombosis, peripheral arterial occlusive disease (PAOD), unstable angina pectoris, myocardial infarction, coronary artery disease, pulmonary embolism, stroke (cerebral thrombosis) due to atrial fibrillation, inflammation and arteriosclerosis. The compounds of the present invention can also be used in the treatment of acute vessel closure associated with thrombolytic therapy and restenosis, e.g. after transluminal coronary angioplasty (PTCA) or bypass grafting of the coronary or peripheral arteries and in the maintenance of vascular access patency in long term hemodialysis patients. Factor Xa inhibitors of this invention may form part of a combination therapy with an anticoagulant with a different mode of action or with a platelet aggregation inhibitor or with a thrombolytic agent. Furthermore, these compounds have an effect on tumor cells and prevent metastases. They can therefore also be used as antitumor agents.

Prevention and/or treatment of thrombotic disorders, particularly arterial or deep vein thrombosis, is the preferred indication.

The invention therefore also relates to pharmaceutical compositions comprising a compound of the present invention and a pharmaceutically acceptable excipient.

The invention likewise embraces compounds of the present invention for use as therapeutically active substances, especially as therapeutically active substances for the treatment and/or prophylaxis of diseases which are associated with the coagulation factor Xa, particularly as therapeutically active substances for the treatment and/or prophylaxis of thrombotic disorders, arterial thrombosis, venous thrombosis, deep vein thrombosis, peripheral arterial occlusive disease, unstable angina pectoris, myocardial infarction, coronary artery disease, pulmonary embolism, stroke due to atrial fibrillation, inflammation, arteriosclerosis, acute vessel closure associated with thrombolytic therapy or restenosis, and/or tumors

In another preferred embodiment, the invention relates to a method for the therapeutic and/or prophylactic treatment of diseases which are associated with the coagulation factor Xa, particularly for the therapeutic and/or prophylactic treatment of thrombotic disorders, arterial thrombosis, venous thrombosis, deep vein thrombosis, peripheral arterial occlusive disease, unstable angina pectoris, myocardial infarction, coronary artery disease, pulmonary embolism, stroke due to atrial fibrillation, inflammation, arteriosclerosis, acute vessel closure associated with thrombolytic therapy or restenosis, and/or tumors, which method comprises administering a compound as defined above to a human being or animal.

The invention also embraces the use of compounds as defined above for the therapeutic and/or prophylactic treatment of diseases which are associated with the coagulation factor Xa, particularly for the therapeutic and/or prophylactic treatment of thrombotic disorders, arterial thrombosis, venous thrombosis, deep vein thrombosis, peripheral arterial occlusive disease, unstable angina pectoris, myocardial infarction, coronary artery disease, pulmonary embolism, stroke due to atrial fibrillation, inflammation, arteriosclerosis, acute vessel closure associated with thrombolytic therapy or restenosis, and/or tumors.

The invention also relates to pharmaceutical compositions containing the compounds of the present for the therapeutic and/or prophylactic treatment of diseases which are associated with the coagulation factor Xa, particularly for the therapeutic and/or prophylactic treatment of thrombotic disorders, arterial thrombosis, venous thrombosis, deep vein thrombosis, peripheral arterial occlusive disease, unstable angina pectoris, myocardial infarction, coronary artery disease, pulmonary embolism, stroke due to atrial fibrillation, inflammation, arteriosclerosis, acute vessel closure associated with thrombolytic therapy or restenosis, and/or tumors. Such pharmaceutical compositions comprise a compound of the present invention.

The inhibition of the coagulation factor Xa by the compounds of the present invention can be demonstrated with the aid of a chromogenic peptide substrate assay as described hereinafter.

Factor Xa activity was measured spectrophotometrically in microtiter plates in a final volume of 150 μl using the following conditions: Inhibition of human factor Xa (Enzyme Research Laboratories) was tested at an enzyme concentration of 3 nM using the chromogenic substrate S-2222 (Chromogenix AB, Mölndal, Sweden) at 200 nM. The reaction kinetics of the enzyme and the substrate were linear with both time and the enzyme concentration. The inhibitors were dissolved in DMSO and tested at various concentrations up to 100 μM. The inhibitors were diluted using HNPT buffer consisting of HEPES 100 mM, NaCl 140 mM, PEG 6000 0.1% and Tween 80 0.02%, pH 7.8. The cleavage of S-2222 by human factor Xa was followed at 405 nm for 5 minutes at room temperature. The velocity of the reaction was determined by the autoreader from the slope of the linear regression fit to 7 time points (1 minute). The initial velocity for each inhibitor concentration was determined by the slope of at least 4 time points in the linear phase by a linear regression fit (mOD/min²). Apparent dissociation constants Kᵢ were calculated according to Cheng and Prusoff [Cheng, Y. C.; Prusoff, W. H. Relationship between the inhibition constant (Kᵢ) and the concentration of the inhibitor that causes 50 percent inhibition (IC₅₀) of an enzyme reaction. Biochem. Pharmacol. 1973, 22, 3099-3108.] based on the IC₅₀ and the respective Kₘ, determined previously (Kᵢ=IC₅₀/(1+S/Kₘ)). The Kₘ for the substrate used was determined under the conditions of the test with at least 5 substrate concentrations ranging from 0.5 to 15 times Kₘ. [Lottenberg R, Hall J A, Blinder M, Binder E P, Jackson C M., The action of thrombin on peptide p-nitroanilide substrates. Substrate selectivity and examination of hydrolysis under different reaction conditions. Biochim Biophys Acta. 1983 Feb. 15; 742(3):539-57]. according to Eadie [Eadie G. S. The inhibition of cholinesterase by physostigmine and prostigmine. J. Biol. Chem. 1942, 146, 85-93.]. The Kₘ for S-2222 amounted to 613

The activity of the low molecular weight substances can, moreover, be characterized in the “prothrombin time” (PT) clotting test. The substances are prepared as a 10 mM solution in DMSO and thereafter made up to the desired dilution in the same solvent. Thereafter, 0.25 ml of human plasma (obtained from whole blood anticoagulated with 1/10 volume of 108 mM Na citrate) was placed in the instrument-specific sample container. In each case 5 μl of each dilution of the substance-dilution series was then mixed with the plasma provided. This plasma/inhibitor mixture was incubated at 37° C. for 2 minutes. Thereafter, they were pipetted to the semi-automatic device (ACL, Automated Coagulation Laboratory (Instrument Laboratory)) 50 μl of plasma/inhibitor mixture in the measurement container. The clotting reaction was initiated by the addition of 0.1 ml of Dade® Innovin® (recombinant human tissue factor combined with calcium buffer and synthetic phospholipids, Dade Behring, Inc., Cat. B4212-50). The time up to the fibrin cross-linking was determined photooptically from the ACL. The inhibitor concentration, which brought about a doubling of the PT clotting time, was determined by fitting the data to an exponential regression (XLfit).

The compounds of the present invention can furthermore be characterised by the Activated Partial Thromboplastin time (aPTT). This coagulation test can e.g. be run on the ACL 300 Coagulation System (Instrumentation Laboratory) automatic analyzer. The substances are prepared as a 10 mM solution in DMSO and thereafter made up to the desired dilution in the same solvent. The test is performed with the Dade® Actin® FS Activated PTT reagent (purified soy phosphatides in 1.0×10⁻⁴ M ellagic acid, stabilizers and preservative, Dade Behring, Inc., Cat. B4218-100) Thereafter, 0.25 ml aliquots of human plasma (obtained from whole blood anticoagulated with 1/10 volume of 108 mM Na citrate) are spiked with 5 μl of test compound in at least 6 concentrations. 50 μl plasma at 4° C. containing 1/50 vol. inhibitor in solvent are incubated with 50 μl Dade® Actin® FS Activated PTT reagent in water at 37° C. for 3 min., then 50 μl CaCl2.2H2O 25 mM in water at 37° C. are added. The time up to the fibrin cross-linking was determined photooptically from the ACL. The inhibitor concentration, which brought about a doubling of the APTT clotting time, was determined by fitting the data to an exponential regression (XLfit).

The Ki values of the active compounds of the present invention preferably amount to about 0.001 to 50 μM, especially about 0.001 to 1 μM. The PT values preferably amount to about 0.5 to 100 μM, especially to about 0.5 to 10 μM. The aPTT values preferably amount to about 0.5 to 100 μM, especially to about 0.5 to 10 μM.

| Example | Ki [μM] factor Xa |
|---|---|
| 1.4 | 0.003 |
| (1S,2R) enantiomer | |

The compounds of the present invention and/or their pharmaceutically acceptable salts can be used as medicaments, e.g. in the form of pharmaceutical preparations for enteral, parenteral or topical administration. They can be administered, for example, perorally, e.g. in the form of tablets, coated tablets, dragées, hard and soft gelatine capsules, solutions, emulsions or suspensions, rectally, e.g. in the form of suppositories, parenterally, e.g. in the form of injection solutions or suspensions or infusion solutions, or topically, e.g. in the form of ointments, creams or oils. Oral administration is preferred.

The production of the pharmaceutical preparations can be effected in a manner which will be familiar to any person skilled in the art by bringing the described compounds of the present invention and/or their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

Suitable carrier materials are not only inorganic carrier materials, but also organic carrier materials. Thus, for example, lactose, corn starch or derivatives thereof, talc, stearic acid or its salts can be used as carrier materials for tablets, coated tablets, dragées and hard gelatine capsules. Suitable carrier materials for soft gelatine capsules are, for example, vegetable oils, waxes, fats and semi-solid and liquid polyols (depending on the nature of the active ingredient, carriers might, however, not be required in the case of soft gelatine capsules). Suitable carrier materials for the production of solutions and syrups are, for example, water, polyols, sucrose, invert sugar and the like. Suitable carrier materials for injection solutions are, for example, water, alcohols, polyols, glycerol and vegetable oils. Suitable carrier materials for suppositories are, for example, natural or hardened oils, waxes, fats and semi-liquid or liquid polyols. Suitable carrier materials for topical preparations are glycerides, semi-synthetic and synthetic glycerides, hydrogenated oils, liquid waxes, liquid paraffins, liquid fatty alcohols, sterols, polyethylene glycols and cellulose derivatives.

Usual stabilizers, preservatives, wetting and emulsifying agents, consistency-improving agents, flavor-improving agents, salts for varying the osmotic pressure, buffer substances, solubilizers, colorants and masking agents and antioxidants come into consideration as pharmaceutical adjuvants.

The dosage of the compounds of the present invention can vary within wide limits depending on the disease to be controlled, the age and the individual condition of the patient and the mode of administration, and will, of course, be fitted to the individual requirements in each particular case. For adult patients a daily dosage of about 1 to 1000 mg, especially about 1 to 300 mg, comes into consideration. Depending on severity of the disease and the precise pharmacokinetic profile the compound could be administered with one or several daily dosage units, e.g. in 1 to 3 dosage units.

The pharmaceutical preparations conveniently contain about 1-500 mg, preferably 1-100 mg, of a compound of the present invention.

The following Examples serve to illustrate the present invention in more detail. They are, however, not intended to limit its scope in any manner.

### EXAMPLES

### Example 1

1.1 3-Oxabicyclo[3.1.0]hexane 2-4-dione (1.0 g; CAS 5617-74-3) was dissolved under an argon atmosphere in THF (30 ml). To this solution 1-(4-amino-3-fluoro-phenyl)-1H-pyridin-2-one (2.0 g; CAS 536747-52-1, prepared according to C. F. Bigge et al., patent application WO 2003045912) were added. The suspension was stirred at r.t. over night, then concentrated. The residue was suspended in 1N HCl. The solid was filtered and washed consecutively with 1N HCl, water and cyclohexane and then dried to give (1RS,2SR)-2-[2-fluoro-4-(2-oxo-2-pyridin-1-yl)-phenylcarbamoyl]-cyclopropanecarboxylic acid (1.88 g) as off-white solid. MS 317.1 ([M+H]⁺)

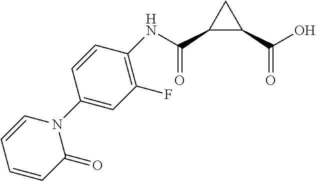

1.2 A suspension of (1RS,2SR)-2-[2-fluoro-4-(2-oxo-2-pyridin-1-yl)-phenylcarbamoyl]-cyclo-propanecarboxylic acid (1.87 g) in MeOH (70 ml) was cooled to 0° C. and then treated with thionylchloride (0.55 ml). The solution was stirred for 3 hrs at 0° C., then concentrated to give (1RS,2SR)-2-[2-fluoro-4-(2-oxo-2-pyridin-1-yl)-phenylcarbamoyl]-cyclopropanecarboxylic acid methyl ester (2.1 g) as light yellow solid which was used in the next reaction step without further purification. MS 329.3 ([M−H]⁻)

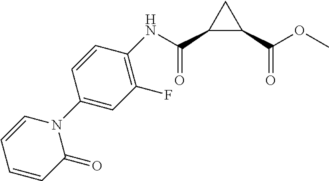

1.3 A solution of 2-amino-5-chloropyridine (3.3. g) in dioxane (30 ml) was treated at r.t. under an argon atmosphere with trimethylaluminium solution (13 ml; 2M in heptane). The reaction mixture was stirred for 2 hrs at r.t. A solution of (1RS,2SR)-2-[2-fluoro-4-(2-oxo-2-pyridin-1-yl)-phenylcarbamoyl]-cyclopropanecarboxylic acid methyl ester (2.1 g) in dioxane (30 ml) was added. The reaction mixture was heated to 100° C. over night. Then, 12 ml water were added. After stirring for 15 min at r.t., Na₂SO₄ was added. Stirring was continued for another 15 min. Then the solid was filtered off and washed with CH₂Cl₂. The filtrate was concentrated. The crude product was purified by chromatography (silica gel; gradient: CH₂Cl₂->CH₂Cl₂/MeOH 95:5) to give (1RS,2SR)-cyclopropane-1,2-dicarboxylic acid 1-[(5-chloro-pyridin-2-yl)-amide] 2-{[2-fluoro-4-(2-oxo-2-pyridin-1-yl)-phenyl]-amide} (2.3 g) as yellow solid. MS 425.0 ([M−H]⁻)

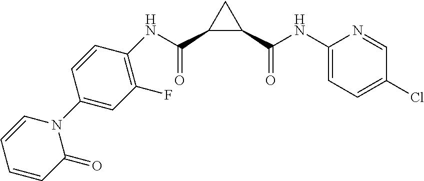

1.4 A solution of (1RS,2SR)-cyclopropane-1,2-dicarboxylic acid 1-[(5-chloro-pyridin-2-yl)-amide] 2-{[2-fluoro-4-(2-oxo-2-pyridin-1-yl)-phenyl]-amide} (442 mg) in CH₂Cl₂/MeOH 2:1 (minimal amount required to obtain a clear solution) was applied to a HPLC system using a Chiralcel OD stationary phase and 30% isopropanol in heptane as eluent. The first eluting enantiomer was concentrated. The residue was triturated with tert-butyl methylether, then filtrated and dried to give (1S,2R)-cyclopropane-1,2-dicarboxylic acid 2-[(5-chloro-pyridin-2-yl)-amide] 1-{[2-fluoro-4-(2-oxo-2H-pyridin-1-yl)-phenyl]-amide} (126 mg) as white solid. Enantiomeric purity: 99% ee.

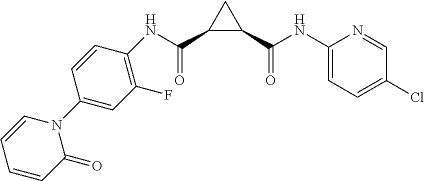

The second eluting enantiomer was isolated by an analogous procedure to give (1R,2S)-cyclopropane-1,2-dicarboxylic acid 2-[(5-chloro-pyridin-2-yl)-amide] 1-{[2-fluoro-4-(2-oxo-2H-pyridin-1-yl)-phenyl]-amide} (118 mg) as white solid. Enantiomeric purity: 78% ee.

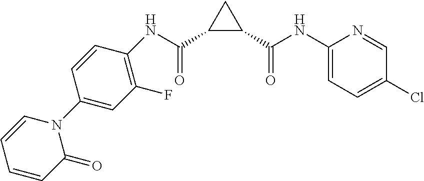

### Example 2

2.1 A solution of 1-methyl-cyclopropane-1,2-dicarboxylic acid dimethyl ester (4.5 g; JAGS 1958, 80, 6568) in MeOH (20 ml) and water (20 ml) was treated with 3.1 g NaOH. The reaction mixture was stirred over night at 50° C., then concentrated. The residual white solid was dissolved in water (25 ml) and washed with diethyl ether (25 ml). The aqueous layer was brought to pH 1 with 3N HCl, then extracted EtOAc. The organic extract was dried (MgSO₄), filtrated and concentrated to give (1SR,2RS)-1-methyl-cyclopropane-1,2-dicarboxylic acid (3.2 g) as white solid which was used in the next reaction step without further purification.

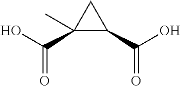

2.2 The starting material (1SR,2RS)-1-methyl-cyclopropane-1,2-dicarboxylic acid (3.2 g) was treated at 0° C. and under an argon atmosphere with trifluoroacetic anhydride. The reaction mixture was stirred at 0° C. for 2 hrs, then concentrated and evaporated three times from THF to give (1SR,5RS)-1-methyl-3-oxa-bicyclo[3.1.0]hexane-2,4-dione (2.9 g) as light yellow liquid which was used in the next reaction step without further purification.

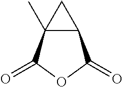

2.3 A solution of (1SR,5RS)-1-methyl-3-oxa-bicyclo[3.1.0]hexane-2,4-dione (2.9 g) in THF (30 ml) was treated at 0° C. and under an argon atmosphere with 2-amino-5-chloropyridine. The reaction mixture (first a solution, then a suspension) was stirred at 0° C. for 1 hr, then at r.t. overnight. The mixture was taken up in water and extracted with EtOAc. The organic layer was dried (MgSO₄), filtrated, concentrated and recrystallized twice from CH₂Cl₂ to give (1SR,2RS)-2-(5-chloro-pyridin-2-ylcarbamoyl)-1-methyl-cyclopropanecarboxylic acid (2.6 g) as white solid. MS 255.3 ([M−H]⁻)

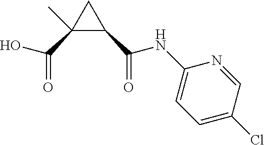

2.4 A solution of (1SR,2RS)-2-(5-chloro-pyridin-2-ylcarbamoyl)-1-methyl-cyclopropanecarboxylic acid (2.2 g) in MeOH (50 ml) was treated at 0° C. and under an Argon atmosphere with thionyl chloride (1.5 ml). The reaction mixture was stirred at r.t. overnight, then concentrated. The crude product was purified by chromatography (silica gel; gradient: CH₂Cl₂->CH₂Cl₂/MeOH 95:5) to give (1SR,2RS)-2-(5-chloro-pyridin-2-ylcarbamoyl)-1-methyl-cyclopropanecarboxylic acid methyl ester (2.1 g) as white solid. MS 269.4 ([M+H]⁺)

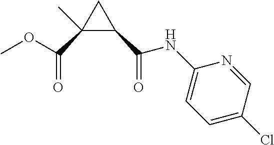

2.5 A solution of 1-(4-amino-3-fluoro-phenyl)-1H-pyridin-2-one (0.61 g; CAS 536747-52-1, prepared according to C. F. Bigge et al., patent application WO 2003045912) in dioxane (4 ml) was treated at r.t. under an argon atmosphere with trimethylaluminium solution (1.49 ml; 2M in heptane). The reaction mixture was stirred for 2 hrs at r.t. A solution of (1SR,2RS)-2-(5-chloro-pyridin-2-ylcarbamoyl)-1-methyl-cyclopropanecarboxylic acid methyl ester (0.2 g) in dioxane (4 ml) was added. The reaction mixture was heated to 100° C. over night, then cooled to r.t. and treated with 0.8 ml H2O. After stirring for 15 min at r.t., Na₂SO₄ was added. Stirring was continued for another 15 min. Then the solid was filtered off and washed with CH₂Cl₂. The filtrate was washed with 1N HCl. The organic layer was dried (MgSO₄) and concentrated. The crude product was purified by chromatography (silica gel; gradient: CH₂Cl₂->CH₂Cl₂/MeOH 95:5) to give (1SR,2RS)-1-methyl-cyclopropane-1,2-dicarboxylic acid 2-[(5-chloro-pyridin-2-yl)-amide] 1-{[2-fluoro-4-(2-oxo-pyridin-1-yl)-phenyl]-amide} (0.175 g) as yellow solid. MS 439.1 ([M+H]⁺)

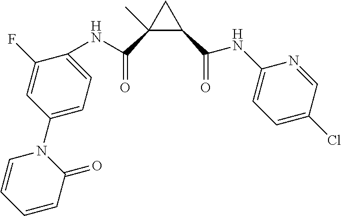

2.6 (1SR,2RS)-1-Methyl-cyclopropane-1,2-dicarboxylic acid 2-[(5-chloro-pyridin-2-yl)-amide] 1-{[2-fluoro-4-(2-oxo-pyridin-1-yl)-phenyl]-amide} (170 mg) was separated into its enantiomers 1-Methyl-cyclopropane-1,2-dicarboxylic acid 2-[(5-chloro-pyridin-2-yl)-amide] (1S,2R)-1-{[2-fluoro-4-(2-oxo-2H-pyridin-1-yl)-phenyl]-amide} and (1R,2S)-1-methyl-cyclopropane-1,2-dicarboxylic acid 2-[(5-chloro-pyridin-2-yl)-amide] 1-{[2-fluoro-4-(2-oxo-2H-pyridin-1-yl)-phenyl]-amide} by preparative HPLC on a Chiralcel OD stationary phase using 20% EtOH in heptane as eluent.

First eluting enantiomer: 45 mg, off-white solid. MS 439.3 ([M−H]⁻)

Second eluting enantiomer: 76 mg, off-white solid. MS 439.3 ([M−H]⁻)

The configuration of both enantiomers is unassigned.

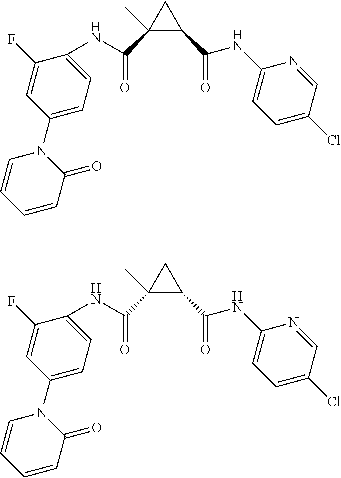

### Example A

Film coated tablets containing the following ingredients can be manufactured in a conventional manner:

| | |
|---|---|
| Ingredients | Per tablet |

| | | | | |
|---|---|---|---|---|
| Kernel: | | | | |
| Compound of the present invention | 10.0 | mg | 200.0 | mg |
| Microcrystalline cellulose | 23.5 | mg | 43.5 | mg |
| Lactose hydrous | 60.0 | mg | 70.0 | mg |
| Povidone K30 | 12.5 | mg | 15.0 | mg |
| Sodium starch glycolate | 12.5 | mg | 17.0 | mg |
| Magnesium stearate | 1.5 | mg | 4.5 | mg |
| (Kernel Weight) | 120.0 | mg | 350.0 | mg |
| Film Coat: | | | | |
| Hydroxypropyl methyl cellulose | 3.5 | mg | 7.0 | mg |
| Polyethylene glycol 6000 | 0.8 | mg | 1.6 | mg |
| Talc | 1.3 | mg | 2.6 | mg |
| Iron oxyde (yellow) | 0.8 | mg | 1.6 | mg |
| Titan dioxide | 0.8 | mg | 1.6 | mg |

The active ingredient is sieved and mixed with microcristalline cellulose and the mixture is granulated with a solution of polyvinylpyrrolidon in water. The granulate is mixed with sodium starch glycolate and magesiumstearate and compressed to yield kernels of 120 or 350 mg respectively. The kernels are lacquered with an aqueous solution/suspension of the above mentioned film coat.

### Example B

Capsules containing the following ingredients can be manufactured in a conventional manner:

| Ingredients | Per capsule | |
|---|---|---|
| Compound of the present invention | 25.0 | mg |
| Lactose | 150.0 | mg |
| Maize starch | 20.0 | mg |
| Talc | 5.0 | mg |

The components are sieved and mixed and filled into capsules of size 2.

### Example C

Injection solutions can have the following composition:

| Compound of the present invention | 3.0 | mg |
|---|---|---|
| Polyethylene Glycol 400 | 150.0 | mg |
| Acetic Acid | q.s. ad pH 5.0 | |
| Water for injection solutions | ad 1.0 | ml |

The active ingredient is dissolved in a mixture of Polyethylene Glycol 400 and water for injection (part). The pH is adjusted to 5.0 by Acetic Acid. The volume is adjusted to 1.0 ml by addition of the residual amount of water. The solution is filtered, filled into vials using an appropriate overage and sterilized.

### Example D

Soft gelatin capsules containing the following ingredients can be manufactured in a conventional manner:

| Capsule contents | | |
|---|---|---|
| Compound of the present invention | 5.0 | mg |
| Yellow wax | 8.0 | mg |
| Hydrogenated Soya bean oil | 8.0 | mg |
| Partially hydrogenated plant oils | 34.0 | mg |
| Soya bean oil | 110.0 | mg |
| Weight of capsule contents | 165.0 | mg |
| Gelatin capsule | | |
| Gelatin | 75.0 | mg |
| Glycerol 85% | 32.0 | mg |
| Karion 83 | 8.0 | mg (dry matter) |
| Titan dioxide | 0.4 | mg |
| Iron oxide yellow | 1.1 | mg |

The active ingredient is dissolved in a warm melting of the other ingredients and the mixture is filled into soft gelatin capsules of appropriate size. The filled soft gelatin capsules are treated according to the usual procedures.

### Example E

Sachets containing the following ingredients can be manufactured in a conventional manner:

| | | |
|---|---|---|
| Compound of the present invention | 50.0 | mg |
| Lactose, fine powder | 1015.0 | mg |
| Microcristalline cellulose (AVICEL PH 102) | 1400.0 | mg |
| Sodium carboxymethyl cellulose | 14.0 | mg |
| Polyvinylpyrrolidon K 30 | 10.0 | mg |
| Magnesiumstearate | 10.0 | mg |
| Flavoring additives | 1.0 | mg |

The active ingredient is mixed with lactose, microcristalline cellulose and sodium carboxymethyl cellulose and granulated with a mixture of polyvinylpyrrolidon in water. The granulate is mixed with magnesiumstearate and the flavouring additives and filled into sachets.

Unless stated to the contrary, all compounds in the examples were prepared and characterized as described. All ranges recited herein encompass all combinations and subcombinations included within that range limit. All patents and publications cited herein are hereby incorporated by reference in their entirety for any purpose.

1. (1S,2R)-cyclopropane-1,2-dicarboxylic acid 2-[(5-chloro-pyridin-2-yl)-amide] 1-{[2-fluoro-4-(2-oxo-2H-pyridin-1-yl)-phenyl]-amide}.

2. (1R,2S)-cyclopropane-1,2-dicarboxylic acid 2-[(5-chloro-pyridin-2-yl)-amide] 1-{[2-fluoro-4-(2-oxo-2H-pyridin-1-yl)-phenyl]-amide}.

3. 1-methyl-cyclopropane-1,2-dicarboxylic acid 2-[(5-chloro-pyridin-2-yl)-amide] (1S,2R)-1-{[2-fluoro-4-(2-oxo-2H-pyridin-1-yl)-phenyl]-amide}.

4. (1R,2S)-1-methyl-cyclopropane-1,2-dicarboxylic acid 2-[(5-chloro-pyridin-2-yl)-amide] 1-{[2-fluoro-4-(2-oxo-2H-pyridin-1-yl)-phenyl]-amide}.

5. A pharmaceutical composition comprising a therapeutically effective amount of a compound of claim 1 and a pharmaceutically acceptable carrier.

6. A pharmaceutical composition comprising a therapeutically effective amount of a compound of claim 2 and a pharmaceutically acceptable carrier.

7. A pharmaceutical composition comprising a therapeutically effective amount of a compound of claim 3 and a pharmaceutically acceptable carrier.

8. A pharmaceutical composition comprising a therapeutically effective amount of a compound of claim 4 and a pharmaceutically acceptable carrier.

9. A method of treating a thrombotic disorder comprising administering to a person in need thereof a therapeutically effective amount of a compound of claim 1.

10. A method of treating a thrombotic disorder comprising administering to a person in need thereof a therapeutically effective amount of a compound of claim 2.

11. A method of treating a thrombotic disorder comprising administering to a person in need thereof a therapeutically effective amount of a compound of claim 3.

12. A method of treating a thrombotic disorder comprising administering to a person in need thereof a therapeutically effective amount of a compound of claim 4.
